(19) United States
(12) Patent Application Publication
(10) Pub. No.: US 2006/0264686 A1
(43) Pub. Date: Nov. 23, 2006

(54) Process for the oligomerization of butenes

(75) Inventors: Lothar Kerker, Duelmen (DE); Armin Rix, Marl (DE); Frank Hoper, Haltern am See (DE); Rainer Malzkorn, Mobile, AL (US); Dirk Roettger, Recklinghausen (DE)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: OXENO OLEFINCHEMIE GMBH, Marl (DE)

(21) Appl. No.: 11/326,522

(22) Filed: Jan. 6, 2006

(30) Foreign Application Priority Data
May 21, 2005 (DE) 102005023549.2-44

(51) Int. Cl. C07C 4/04

(52) U.S. Cl. 585/535

(57) ABSTRACT

The invention relates to a process for the oligomerization of butenes, in which a stream which contains predominantly butenes and has been obtained by separation from a stream of hydrocarbons having a lower content of butenes, scrubbing and drying is fed to the oligomerization.

### REFERENCE TO RELATED APPLICATIONS

This application claims priority to German patent application 102005023549.2-44 filed May 21, 2005, incorporated herein by reference.

### FIELD OF THE INVENTION

The invention relates to a process for the oligomerization of butenes from a hydrocarbon stream which comprises butenes and that has preferably been obtained by separation from a stream of hydrocarbons having a lower content of butenes, scrubbing and subsequent drying.

### BACKGROUND OF THE INVENTION

The oligomerization of olefins, in particular C₄-olefins, is a process which is frequently employed in industry. The oligomerization of C₄-olefins gives, in particular, olefins having eight, twelve, sixteen or twenty carbon atoms. These olefins are used, for example, for preparing plasticizer alcohols (e.g., C₉- or C₁₃-alcohols) or alcohols (e.g., C₁₃—, C₁₇- or C₂₁-alcohols) for preparing detergent raw materials.

Various oligomerization processes are known. In principle, there are three process variants. Oligomerization over acid catalysts (process A), in which, for example, zeolites or phosphoric acid on supports are/is used industrially, has been known for a long time. This gives isomer mixtures of branched olefins which are essentially dimethylhexenes (WO 92/13818). Another process which is likewise practiced worldwide is oligomerization using soluble Ni complexes, known as the Dimersol process (process B) (B. Cornils, W. A. Herrmann, Applied Homogenous Catalysis with Organometallic Compounds, pages 261-263, Verlag Chemie 1996). Finally, mention may be made of oligomerization over fixed-bed nickel catalysts, e.g. the process of OXENO Olefinchemie GmbH. The process has become known in the literature as the Octol process (process C) (Hydrocarbon Process., Int. Ed. (1986) 65 (2. Sect. 1), pages 31 to 33) and may also be found in DE 39 14 817 and EP 1 029 839.

The known oligomerization processes have the disadvantage that the olefin conversion in the oligomerization stage is not complete and is frequently only in the region of about 50%. To improve the conversion, either a plurality of oligomerization stages are connected in series or the oligomerization stage is carried out as a loop process or with (partial) recirculation of the product stream. These processes have the disadvantage that large streams of material have to be moved. This is disadvantageous particularly when not only the butenes to be reacted but also butanes which do not undergo oligomerization are present in the oligomerization mixture.

Another disadvantage of the oligomerization processes known from the prior art is the sensitivity of the catalysts used to catalyst poisons present in the feedstock. The catalyst poisons differ depending on the oligomerization process used (variants A, B or C). Methods of removing catalyst poisons by use of adsorbents are described, for example, in DE 19845857 and DE 3914817. These are particularly applicable when using transition metal catalysts, usually catalysts based on nickel.

The separation of butenes and butanes is known in the specialist literature. Since a simple separation by distillation is not industrially practical because of the boiling point differences, extractive distillations with polar solvents are usually employed for this purpose. Thus, EP 501 848 describes the fractionation of a butadiene-free C₄ fraction by extractive distillation with an extractant such as N-methylpyrrolidone (NMP) or dimethylformamide (DMF) in three stages: in the first stage, the C₄-hydrocarbon feed mixture is admixed with the extractant in an extractive distillation column. Here, the olefinic constituents are dissolved in the extractant, so that the less soluble aliphatic constituents can be separated off. To achieve further separation or to recover the extractant, partial desorption of the butenes from the extract is then carried out under a pressure of from 0.4 to 0.5 MPa. To recover the remaining extractant, the extract is subsequently boiled at atmospheric pressure and a temperature of from 140 to 170° C.

JP 692 876 discloses the use of dimethylformamide as polar extractant for butene/butane separation. This document also states that after the extractive distillation and the separation of the aliphatic constituents from the hydrocarbon feed mixture, the major part of the polar extractant is recovered by means of a desorption stage at from 1 to 2 atmospheres with recirculation of the major part of the extractant. The butene-containing fraction is freed of the butenes at an elevated pressure of from 1 to 15 atmospheres in a purification stage; the pure extractant obtained in this way is once again recirculated to the extractive distillation stage.

According to the examples, the extractant still contains large amounts of butenes which are recirculated, i.e. conveyed in a circuit, together with the extractant. This is energetically and economically unfavorable.

In Bender, D.; Lindner, A.; Schneider, K. J; Volkamer, K.; BASF ENTWICKLUNGSARBEITEN AM BUTADIENVERFAHREN DER BASF; Erdoel Kohle, Erdgas, Petrochem. (1981) 34(8), 343, Bender et al. describe the use of water-containing extractants in processes for the separation of saturated and unsaturated hydrocarbons. The selectivity of the extractant can be optimized in this way. Increasing the boiling pressure of the extractant by addition of water can be advantageous in industry in order to be able, for example, to condense the vapor by means of normal cooling water/river water without compression of the vapor.

The use of extractants consisting of mixtures of one or more polar organic solvents and water leads to the problem that proportions of the extractant are obtained both at the top of the extraction column and the top of the degasser column. This is mostly water in which small proportions of the organic solvents are usually still present. In the known processes, these amounts of extractant are discharged from the process together with the C₄-hydrocarbons, which in continuous operation leads to a change in the composition of the extractant.

DE 102 42 923 describes a process variant for the separation of butenes and butanes which partly solves this problem. Here, the separation of the butenes from the polar, water-containing extractant is carried out in two stages. A further stage is required to separate off the aqueous phase. In this, the butenes are obtained as bottom fraction, while an aqueous phase is obtained in a decanter at the top of the column and this is recirculated to the first stage of the separation of butenes and extractant.

DE 2 359 300 describes the use of decanters for separating off an aqueous phase in a process for the recovery of saturated hydrocarbons from hydrocarbon mixtures. The extractive distillation is in this case carried out with reflux of water from the top of the column. An order of magnitude of polar impurities of 100 ppm for the saturated hydrocarbons obtained in this processing is reported.

DE 102 19 375 describes a process for the recovery of butenes from a C₄ fraction, in which an attempt has been made to improve the utilization of energy in the process. The heat present in the bottom product from the stage of separation of the extractant (degassing zone) is employed for heating a substream which is taken off from the degassing zone before it is recirculated to the degassing zone.

### SUMMARY OF THE INVENTION

It is an object of the present invention to provide a process by which the disadvantages of the processes of the prior art can be largely avoided. In particular, it is an object of the present invention to provide a process for the oligomerization of butenes, in which the raw material used has a sufficient butene concentration and is at the same time largely free of catalyst poisons.

It has surprisingly been found that combination of a butene/butane separation based on an extractive distillation with an oligomerization makes it possible to avoid the disadvantages of the prior art in a simple manner. In particular, the proportion of catalyst poisons can also be reduced when the butene-enriched stream from the extractive distillation is scrubbed with water or an aqueous solution prior to drying for use in the oligomerization.

### BRIEF DESCRIPTION OF THE FIGURES

FIG. 1 shows the extraction column K1, the column K2 (degasser) and the solvent circuit of the extractant (stages a) and b)).

FIG. 2 shows one possible way of carrying out the scrubbing (stage c) of the distillate D2.

FIG. 3 shows how the distillate D1 from the extraction column K1 can be purified in the same way as the distillate D2 from column K2.

FIG. 4 shows an additional purification by distillation.

FIG. 5 shows a possible way of connecting the column K7 to the columns K1 and K2.

FIG. 6 shows an alternative way of integrating the C₅ removal into the process.

FIG. 7 shows one possible way of working up the stream D1 to give high-purity n-butane.

FIG. 8 shows an alternative to the purification of the n-butane shown in FIG. 7.

FIG. 9 shows a possible embodiment of the process of the invention which is obtained by combining the plants described in FIG. 2, FIG. 3 and FIG. 5 (stages a), b), c), d), e), f) and j)).

FIG. 10 schematically shows a variant of the process of the ivention in which butene-containing streams separated off in the oligomerization are recirculated to stage a).

FIG. 11 shows a variant of the process of the invention in which the butene-containing streams separated off in the oligomerization are recirculated to stage a).

FIG. 12 schematically shows an integrated plant in which the process of the invention has been combined with a unit for the preparation of oligomers and of high-purity n-butane.

FIG. 13 shows how recirculation of the butene-rich stream S4 is preferably effected downstream of the C₅ separation.

### DETAILED DESCRIPTION OF THE PREFERRED EMBODIMENTS

The present invention accordingly provides a process for the oligomerization of butenes in the presence of a transition metal catalyst, in which a hydrocarbon stream which is entirely or partly a hydrocarbon stream (1) which comprises predominantly butenes is used as feedstock where hydrocarbon stream (1) has been obtained by separation from a different stream of hydrocarbons (hydrocarbon stream 2) having a lower content of butenes. In a preferred embodiment hydrocarbon stream (1) is obtained by
a) extractive distillation of hydrocarbon stream (2) comprising saturated and unsaturated C₄-hydrocarbons with a polar extractant to give an overhead fraction (3) which is enriched in saturated hydrocarbons and a bottom fraction (4) which is enriched in unsaturated hydrocarbons and comprises the polar extractant,
b) separation of the bottom fraction (4) by distillation to give an overhead fraction (5) which comprises butenes and a bottom fraction (6) which comprises the polar extractant,
c) scrubbing of at least part of the overhead fraction (5) with water or an aqueous solution and
d) drying of the part of the overhead fraction (5) which has been treated in step c) to give a substantially water-free butene-containing hydrocarbon stream (1).

The process of the invention has the advantage that butenes present in hydrocarbon streams can be converted in high yield into oligomers in a simple manner. The separation of the saturated hydrocarbons from the butenes makes the streams to be treated in the subsequent oligomerization significantly smaller, which enables energy costs and materials costs to be saved.

The process of the invention has the additional advantage that the transition metal catalyst used in the oligomerization has a relatively long operating life, since catalyst poisons are at least partly removed by the scrub. This is of particular importance in the case of heterogeneous catalysts. In the case of these, the operating life should be sufficient to enable the process to be carried out in fixed-bed reactors (e.g. adiabatic fixed-bed reactors, shell-and-tube reactors).

The energy necessary for operation of the process can be reduced by means of suitable ways of carrying out the process, as are described in preferred embodiments of the process of the invention. For example, if, in the process of the invention the bottom fraction from stage b) is at least partly fed as extractant into stage a) and the heat energy present in the bottom fraction is utilized in a heat exchanger to heat the feed to the distillation column of stage b), the amount of energy necessary for operation of the process can be significantly reduced compared to conventional processes.

In addition, the heating of the stream fed to stage b) enables partial vaporization of the constituents of the stream to be achieved, as a result of which improved separation can be achieved in stage b). In particular, this embodiment of the process of the invention has the advantage that the capital costs can be kept lower, since pumps and collection trays can be dispensed with and the column of stage b) can be constructed with a lower column height.

The use of decanters in a preferred embodiment of the process of the invention has the advantage that the extractants can largely be removed again from the hydrocarbons to be separated after the separation into unsaturated and saturated hydrocarbons. As a result of at least partial recirculation to the extraction stage a) or the separation stage b), the consumption of solvent is reduced and the composition of the extractant mixture also remains largely constant in continuously operating processes. The use of the decanter technology which is preferred according to the invention in step b) is therefore advantageous even though the unsaturated hydrocarbon streams are purified further by after-treatment in a water scrub. The scrub with water or aqueous solutions makes it possible to reduce the content of the organic extractant constituent to such an extent that the butene-containing stream can, after drying, be used in the oligomerization.

A scrub with water or aqueous solutions can also be utilized for after-purification of the overhead fraction (3) obtained in step a). This can be necessary for some applications of the resulting butanes or butenes. For example, butanes which can be used as fuel can be obtained from the overhead fraction (3) after further work-up. Even low residual concentrations of extractant in them cause a perceptible, unpleasant odor which prevents use of the butanes as fuel. Removal of amounts of extractant in the decanters used according to the invention prior to a water scrub can additionally counteract introduction of extractant into the water scrubs.

In the process of the present invention a hydrocarbon stream which is to be used for oligomerization is firstly enriched in butenes by extractive distillation and subsequently depleted in any catalyst poisons present before the hydrocarbon stream is fed to the oligomerization.

In a preferred embodiment of the process of the invention for the oligomerization of butenes in the presence of a transition metal catalyst, preferably a heterogeneous transition metal catalyst, in which a hydrocarbon stream which consists entirely or partly of a hydrocarbon stream (1) which comprises predominantly butenes and has been obtained by separation from a stream of hydrocarbons (2) having a lower content of butenes is used as feedstock, the hydrocarbon stream (1) is obtained by
a) extractive distillation of a stream (2) comprising saturated and unsaturated C₄-hydrocarbons with a polar extractant to give an overhead fraction (3) which is enriched in saturated hydrocarbons and a bottom fraction (4) which is enriched in unsaturated hydrocarbons and comprises the polar extractant,
b) separation of the bottom fraction (4) by distillation to give an overhead fraction (5) which comprises butenes as unsaturated hydrocarbons and a bottom fraction (6) which comprises the polar extractant,
c) scrubbing of at least part of the overhead fraction (5) with water or an aqueous solution and
d) drying of the part of the overhead fraction (5) which has been treated in step c) to give a substantially water-free butene-containing hydrocarbon stream (1).

The oligomerization of the butenes present in the hydrocarbon feed stream can be carried out according to one of the processes known from the prior art. In the process of the invention, the processes known under the names of Dimersol process (B. Cornils, W. A. Herrmann, Applied Homogeneous Catalysis with Organometallic Compounds, pages 261 to 263, Verlag Chemie, 1996) or Octol process (EP 1 029 839, DE 39 14 817, Hydrocarbon Processing, International Edition (1986), 65(2, Sect. 1), 31 to 33) are preferably used as oligomerization process. The documents cited here are expressly incorporated by reference and their disclosure is part of the disclosure of the present invention. In the Dimersol process, a dissolved nickel catalyst is used. In the Octol process, it is possible to use heterogeneous catalysts which can comprise transition metals, mainly nickel. For the purposes of the present invention, particular preference is given to using heterogeneous nickel catalysts, in particular supported nickel catatlysts such as nickel on silicon dioxide or nickel on silicon dioxide-aluminum oxide, for the oligomerization. Further heterogeneous nickel catalysts are described, for example, in U.S. Pat. No. 5,169,824, EP 1 268 370 and WO 95/14647 and in the references cited there. The nickel content of the catalysts is typically from 1 to 25% by mass.

The oligomerization in the process of the invention can, in particular, be carried out as described below. In this process, the butenes present in the hydrocarbon stream which is at least partly made up of the hydrocarbon stream (1) are oligomerized over a nickel-containing catalyst at temperatures of from 0 to 200° C. and pressures of from 0.1 to 7 MPa. The oligomerization can be carried out either in the presence of homogeneous catalysts or in the presence of heterogeneous catalysts. The oligomerization is preferably carried out over a heterogeneous nickel-containing catalyst, particularly preferably over a fixed bed of nickel-containing catalyst and very particularly preferably over a fixed bed of nickel-, silicon- and aluminum-containing catalyst. The oligomerization can be carried out in the liquid phase, in the gas/liquid mixed phase or in the gas phase. The oligomerization is preferably carried out in the liquid phase.

The process of the invention is used for preparing oligomers from a hydrocarbon stream (1) comprising predominantly butenes. This hydrocarbon stream (1) preferably comprises more than 50% by mass, preferably at least 60% by mass and more preferably at least 70% by mass and particularly preferably at least 80% by mass, of butenes.

In addition to the hydrocarbon stream (1) obtained according to the invention, the hydrocarbon feed stream used in the oligomerization can comprise one or more further hydrocarbon streams, in particular C₄-hydrocarbon streams. The hydrocarbon stream or streams which are additionally used preferably comprise at least 50% by mass, more preferably from 60 to 85% by mass, of butenes. As additional hydrocarbon streams, it is possible to use, for example, streams obtained in the work-up of C₄ fractions of an FC cracker (fluid catalytic cracker), hydrocarbon streams which are obtained in the work-up of C₄ fractions from steam crackers and/or hydrocarbon streams which have been obtained wholly or partly as streams in an oligomerization process by separating off the product of the oligomerization and comprise the C₄-hydrocarbons which have not been reacted in the oligomerization.

A stream (2) comprising saturated and unsaturated C₄-hydrocarbons is preferably used as feed in step a) in the process of the invention. This stream comprises preferably n-butane and optionally isobutane as saturated hydrocarbons and 1-butene, cis-2-butene and/or trans-2-butene as unsaturated hydrocarbons. In addition to the butanes and butenes to be separated, further hydrocarbons which have a larger or smaller number of carbon atoms than the C₄-hydrocarbons to be separated can be present in the hydrocarbon stream (2). The hydrocarbon stream (2) preferably comprises the C₄-hydrocarbons to be separated together with hydrocarbons which have a maximum of ten, preferably 5, 4, 3 or 2 and very particularly preferably 1, more carbon atom(s) or 3, 2 or 1 less carbon atom(s) than the hydrocarbons to be separated. Preference is given to using hydrocarbon streams (2) comprising butanes, in particular n-butane and/or isobutane, as saturated hydrocarbons and 1-butene, cis-2-butene and/or trans-2-butene and possibly isobutene and optionally 1,3-butadiene as unsaturated hydrocarbons in step a) in the process of the invention. Streams consisting almost entirely of n-butane, cis-butene, trans-butene and 1-butene can particularly advantageously be used as hydrocarbon streams (2) in the process of the invention. Further components such as isobutane, isobutene, C₁-C₃— and C₅₊-hydrocarbons are preferably present therein in a proportion of less than 5% by mass, particularly preferably less than 2% by mass. The hydrocarbon streams (2) to be used in step a) preferably comprise at least 50% by mass, more preferably at least 75% by mass and particularly preferably 95% by mass, of butanes and butenes. Particular preference is given to using a hydrocarbon stream (2) comprising from 5 to 75% by mass, preferably from 10 to 65% by mass and particularly preferably from 15 to 50% by mass, of butenes in stage a).

If C₅-hydrocarbons and higher hydrocarbons (C₅₊-hydrocarbons) are present in the hydrocarbon streams used, it can be advantageous to separate off all or part of these by distillation before entry into stage a), from the overhead fraction from stage a) and/or from the overhead fraction from stage b), if appropriate after going through further process steps.

Suitable feedstocks for use in step a) of the process of the invention are, for example, hydrocarbon streams which are obtained in the work-up of C₄ fractions from an FC cracker, hydrocarbon streams which are obtained in the work-up of C₄ fractions from steam crackers and/or hydrocarbon streams which have been obtained wholly or partly as streams in an oligomerization process by separating off the product of the oligomerization and comprise the C₄-hydrocarbons which have not been reacted in the oligomerization.

The C₄ fractions from FC crackers generally comprise from 20 to 70% by mass of butanes and from 3.0 to 80% by mass of butenes. Any balance can comprise other C₃-C₅-hydrocarbons. A typical composition of a C₄ fraction from an FC cracker is as follows:

| | | |
|---|---|---|
| Propane | 0.3% | by mass |
| Propene | 1.2% | by mass |
| n-Butane | 12% | by mass |
| i-Butane | 30% | by mass |
| 1-Butene | 14% | by mass |
| i-Butene | 10% | by mass |
| trans-2-Butene | 16% | by mass |
| cis-2-Butene | 14% | by mass |
| 1,3-Butadiene | 0.5% | by mass |
| C₅-hydrocarbons | 2% | by mass |

The butadiene present in the C₄ fraction from FC crackers and any acetylenically unsaturated compounds present should preferably be removed before use in the process of the invention. They are preferably removed from the C₄ fraction by selective hydrogenation, e.g. as described in EP-B 0 081 041 and DE-C 15 68 542, particularly preferably by selective hydrogenation to a residual content of less than 5 ppm by mass. The isobutene present can optionally be removed before use in the process of the invention, for example by etherification with alcohols, in particular methanol or ethanol, or by means of other selective chemical reactions, for example with water to form tert-butanol.

The mixture of C₄-hydrocarbons which is obtained after selective hydrogenation of the multiply unsaturated hydrocarbons, removal of isobutene and isobutane and comprises mainly 1-butene, 2-butene and n-butane is a preferred feedstock to be used as hydrocarbon stream (2) in the process of the invention.

Further preferred hydrocarbon streams which are particularly suitable as hydrocarbon stream (2) for use in step a) of the process of the invention are C₄-hydrocarbon streams obtained in the work-up of C₄ fractions from steam crackers. The multiply unsaturated compounds which may be present are separated off therefrom or selectively hydrogenated. The isobutene can be separated off from the remaining mixture by means of selective chemical reactions, for example to form tert-butyl ethers such as methyl tert-butyl ether or ethyl tert-butyl ether, or to form tert-butanol. The mixture of C₄-hydrocarbons obtained in this way comprises mainly 1-butene, 2-butenes, n-butane and isobutane and is often referred to as raffinate II in industry. Isobutane and 1-butene can be separated off, either completely or partly, from the raffinate II by distillation. The mixture which remains, viz. raffinate III, usually comprises n-butenes and n-butane and is a particularly preferred feed mixture for use as hydrocarbon stream (2) in step a) of the process of the invention.

A further preferred feed mixture for use as hydrocarbon stream (2) in step a) of the process of the invention is a C₄-hydrocarbon stream which is obtained as stream from a process for the oligomerization of butenes after the product of the oligomerization has been separated off and comprises the C₄-hydrocarbons which have not been reacted in the oligomerization.

Industrially operated oligomerizations of n-butenes from mixtures of n-butenes and butane give dimers, trimers and higher oligomers. Since complete conversion of the butenes is usually not practical from an economic point of view, a mixture of n-butenes and butane which cannot be economically fractionated purely by distillation is obtained. This mixture represents a further preferred feed mixture for use as hydrocarbon stream (2) in step a) of the process of the invention. The process of the invention makes it possible for the butenes still present to be utilized in an oligomerization. The process additionally produces a butane-rich fraction from which, for example, high-purity butane which is essentially free of substances which alter the odor can be obtained. The oligomerization from which the C₄-hydrocarbon streams used as hydrocarbon stream (2) in stage a) are obtained can be completely or partially identical to the oligomerization in which the butenes present in the hydrocarbon stream (1) are reacted.

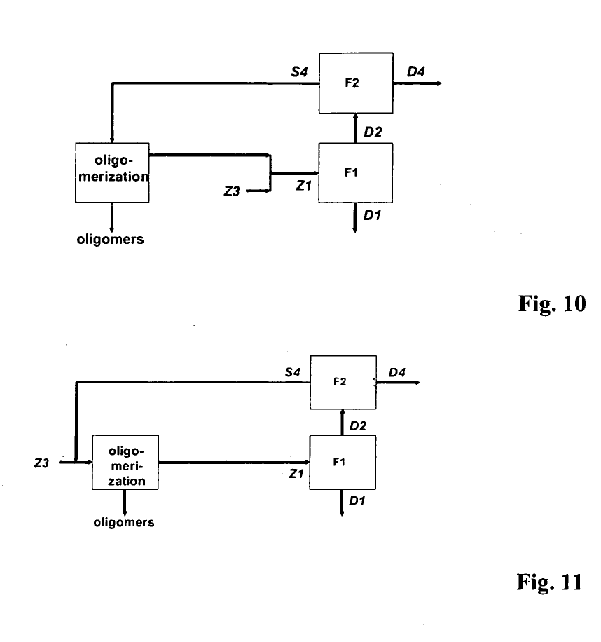

In a very particularly preferred variant of the process of the invention, a mixture which has been obtained wholly or partly as stream in the oligomerization of the invention after the product of the oligomerization has been separated off and comprises the C₄-hydrocarbons which have not been reacted in the oligomerization can be used as hydrocarbon stream (2) in stage a). Such a preferred variant of the process of the invention is shown schematically in FIG. 10 to FIG. 13.

As can also be seen from the figures indicated, the process of the invention can be utilized particularly advantageously when only part of the butenes, preferably only from 60 to 90%, is reacted in the oligomerization and the unreacted butenes are, after the oligomerization products have been separated off, at least partly reused in stage a).

For feeding a hydrocarbon stream (2) to stage a) of the process of the invention, there are three preferred possibilities:
1) In this case, stage a) is carried out using a hydrocarbon stream (2) which is a mixture of a hydrocarbon stream which has been obtained from an oligomerization after oligomers have been separated off and comprises butenes and an external hydrocarbon stream. This option is particularly preferred in the case of external hydrocarbon streams (streams which do not come from the process of the invention) having low butene contents, for example from the work-up of fractions from FC crackers.
2) Stage a) is carried out using exclusively the butenes originating from the oligomerization after the oligomers have been separated off. The introduction of the external hydrocarbon streams into the oligomerization can be effected together with the hydrocarbon stream (1) obtained from stage d) or can be effected at another point of the oligomerization, in the case of multistage oligomerization processes, for example in different stages.
3) The external hydrocarbon stream is fed together with the overhead fraction (5) into stage d) of the process of the invention where it is dried together with the overhead fraction (5). In a specific embodiment, this drying is carried out as described below in a drying column from which water and, if appropriate, a hydrocarbon fraction (for example isobutane, 1-butene) are taken off at the top.
   It can be advantageous for at least part of the bottom fraction from stage b) to be fed as extractant into stage a). Preference is given to all or virtually all of the bottom fraction from stage b) being recirculated as extractant to stage a). To enable foreign substances (for example decomposition products of the extractant) which accumulate in the recycle stream of the extractant to be removed from the process, it can be advantageous for a substream to be discharged continuously or batchwise and either regenerated or replaced by fresh extractant. To even out fluctuations in the process, it can be advantageous to provide a buffer vessel or storage vessel for the extractant.

In a preferred embodiment of the process of the invention, at least part of the bottom fraction from stage b) is fed as extractant into stage a) and the heat energy present in the bottom fraction is utilized in a heat exchanger for heating the feed to the distillation column for stage b).

In the process of the invention, the distillation in stage a) or stage b) is preferably carried out in an apparatus comprising a decanter and the condensed overhead fraction from the distillation is separated in this decanter into a nonpolar stream comprising hydrocarbons and a polar stream comprising the extractant. For this purpose, the streams obtained as vapor at the top of the column are firstly treated so that the stream or part thereof is present as a liquid phase. The treatment can, for example, be carried out by means of cooling with or without prior compression, so that at least part of the stream is obtained as a liquid phase.

In the process of the invention, it can be advantageous for both the extractive distillation in stage a) and the separation by distillation in stage b) to be carried out in an apparatus comprising a decanter. If a plurality of apparatuses are present for carrying out a plurality of stages a) and/or b), it is possible for all or only some of the apparatuses to be equipped with a decanter.

The polar and possibly aqueous stream obtained from the decanter or decanters can be at least partly recirculated to the process, either directly or after work-up. Recirculation can be to stage a), in this case preferably into the feed stream of extractant, or to stage b) or to a stock vessel for the extractant which is usually present. If a plurality of stages a) and/or b) are present, it can be advantageous to recirculate the polar stream from the decanter or decanters to the first stage of stage a). The polar, aqueous stream obtained from the decanter or decanters is preferably fed at least partly into the stock vessel for the polar extractant. In this way, additional pumps can be dispensed with.

The extractive distillation of stage a) is preferably operated at a pressure of from 0.2 to 1.5 MPa and a temperature of from 40 to 100° C. Such columns are usually operated in countercurrent, i.e. the extractant is introduced into the column at a point above the point at which the stream to be extracted is introduced. The stream to be extracted (viz. the C₄ stream to be treated) is preferably introduced into the middle third of the column. In the present case, the C₄-hydrocarbon stream to be extracted is preferably vaporized prior to introduction into the column and is, as a gaseous stream, brought into contact with the polar extractant in a mass ratio of from 15:1 to 6:1, preferably from 12:1 to 6:1 (gas:liquid). The column is advantageously equipped with internals or packing to produce a very large exchange area. Internals which have been found to be useful for the extractive distillation column are, in particular, packing, bubble cap trays or valve trays. A preferred extraction column has from 10 to 50, preferably from 15 to 25, theoretical plates. The extraction column used according to the invention in stage a) is preferably operated at a trickle density of from 10 to 100 m³/(m²*h), preferably from 40 to 60 m³/(m²*h).

Above the point at which the extractant is fed in, a distillation section is preferably provided in the column so that the proportion of extractant in the overhead product from the column is reduced. This section preferably extends to the top of the column. Here too, internals, ordered packing or random packing, which can be identical to or different from those in the column section below the extractant inlet, are advantageously installed in the upper column section. In addition, it can be useful for this column section to have a smaller diameter than the lower column section, for example to optimize the trickle density.

At the top of the column, the mixture comprising mainly butanes can be taken off in gaseous form and passed to a further use. The overhead product from the extractive distillation of stage a) is, however, preferably condensed, e.g. by cooling with or without prior compression, and transferred to a decanter and there separated into a polar stream and a nonpolar stream. The polar stream, which comprises residual extractant, can, for example, be recirculated to the feed to the column or else be utilized in another way.

The nonpolar stream which is obtained at the top of the column of stage a) contains less than 40% by mass of unsaturated C₄-hydrocarbons, preferably less than 25% by mass, particularly preferably less than 15% by mass. All or part of it can be passed to a further work-up, e.g. a work-up by distillation. Part can be recirculated as runback to the column of stage a). Preference is given to providing a runback stream, with a reflux ratio of from 0.05 to 17 [kg/kg], more preferably from 0.5 to 4 [kg/kg], defined as the ratio of the amount of nonpolar stream recirculated to the column to the amount of nonpolar stream discharged, preferably being set.

The work-up of the nonpolar, butane-containing stream can, for example, be carried out by scrubbing it with water or an aqueous solution, e.g. in a scrubbing column (stage e)). The nonpolar stream saturated with water which is obtained in this scrub in stage e) or else the nonpolar stream which is obtained at the top of the column of stage a) can then be worked up in a further distillation column to give a virtually water-free nonpolar stream (stage f)). For this purpose, this distillation column can likewise have a decanter in which the condensed overhead product is separated into an organic phase, of which from 50 to 100% by mass is returned to the column, and an aqueous phase which is discharged. The stream which is not returned to the column, which comprises low boilers such as isobutane, can be used further in another way. The bottom product obtained in this distillation column has a water content of less than 50 wppm (ppm by mass), particularly preferably less than 5 wppm. In addition, when part of the organic phase of the overhead product is discharged, a bottom product which is depleted in low boilers, for example isobutane, compared to the starting material introduced into stage f) is obtained. The distillation column of stage f) is preferably operated at a temperature at the top of from 40 to 60° C., particularly preferably a temperature at the top of from 45 to 55° C.

Polar impurities which, in particular, influence the odor of saturated hydrocarbons such as butanes can be very substantially removed from the overhead product from stage a) by means of the scrub and/or subsequent removal of the water in these optional stages e) and/or f).

A further possible way of working up the nonpolar stream obtained at the top of the column of stage a) or a nonpolar stream as is obtained from the optional stages e) and/or f), preferably the butane-rich bottom product obtained from stage f), can comprise removal of any unsaturated hydrocarbons still present in an optional hydrogenation stage g). In this stage, any olefins still present in the nonpolar stream obtained at the top of the column of stage a) or from the optional stages e) and/or f) can be converted into alkanes. The hydrogenation stage g) can, in the simplest case, comprise a hydrogenation reactor in which the unsaturated olefins are converted into alkanes according to the prior art. The hydrogenation stage can optionally further comprise one or more distillation columns in which the product obtained from the hydrogenation reactor is separated further by distillation. For example, C₁-C₃-hydrocarbons and/or hydrocarbons having 5 or more carbon atoms can be separated off in such a distillation column. If both n-butane and i-butane are present in the butanes, these can be separated into the pure substances by distillation.

To remove traces of further impurities, it can be advantageous for the nonpolar stream obtained at the top of the column of stage a) or a nonpolar stream as is obtained from one or more of the optional stages e) and/or f) and/or g) to be additionally or alternatively passed to an after-purification (optional stage h)) in which a treatment with one or more adsorbents, e.g. in adsorbent beds, is carried out. Customary adsorbents are, for example, activated carbon and molecular sieves.

The nonpolar stream obtained at the top of the column of stage a) is particularly preferably firstly scrubbed in a stage e) and subsequently dried in a stage f). The dried nonpolar stream from f) is subsequently passed to a hydrogenation stage g) and then treated with an adsorbent in stage h). In this way, the butane-containing nonpolar streams which are obtained at the top of the column of stage a) can be purified to give high-purity n-butane.

An alternative, preferred process variant can be realized by inserting a hydrogenation stage i) between process stages e) and f). In this variant of the process of the invention, the product from the scrub in stage e) is firstly fed into a hydrogenation reactor in which unsaturated hydrocarbons are hydrogenated to saturated hydrocarbons by methods known per se. The product from this hydrogenation is then passed directly to the distillation step of stage f), in which the saturated hydrocarbon is obtained as bottom product. An advantage of this arrangement can be that full reflux is not set at the top of the column, but C₁-C₃-hydrocarbons and isobutane can instead be simply separated off as substream.

The extractant and in particular the unsaturated hydrocarbons from the mixture to be fractionated accumulate at the bottom of the column of stage a). The bottom of the column is preferably heated externally.

The bottom fraction obtained from stage a) is fed into the distillation column of stage b). The bottom fraction is preferably heated by heat exchange with steam or another heating medium, preferably by indirect heat exchange with the bottom fraction from stage b), before being introduced into the distillation column of stage b). If the heat energy present in the bottom fraction when using the bottom fraction from stage b) as heating medium is not sufficient to transfer the desired quantity of heat energy, it is possible to provide further heat exchangers in which the stream fed to stage b) can be heated by means of other heating media such as steam or other process streams. The feed to the column of stage b) is preferably introduced into the upper half, particularly preferably in the upper third, of the column of stage b).

In a preferred embodiment of the process of the invention, the feed to the distillation column of stage b) can be heated under a pressure which is higher than the pressure in the distillation column b). In this embodiment, the feed is depressurized into the distillation column of stage b) after heating.

In a further preferred embodiment of the process of the invention, the feed to the distillation column of stage b) can be heated under a pressure which corresponds to the pressure in the distillation column b). In this case, the feed is preferably heated to such an extent that at least part of it vaporizes. It can here be advantageous to separate the feed to the distillation column of stage b) into a vapor phase and a liquid phase before it enters the column and to introduce these phases individually onto different or identical, preferably different, trays of the distillation column of step b). The gaseous phase is particularly preferably introduced from 1 to 5 theoretical plates above the point at which the liquid phase is fed in. A preferred embodiment of an apparatus for heating the feed is, for example, known as a kettle vaporizer in the technical literature.

Stage b) is preferably operated at a pressure of from 0.1 to 1 MPa, preferably from 0.3 to 0.5 MPa, and a temperature of from 120 to 230° C., preferably from 125 to 190° C. The bottoms from the column can be recirculated to the extractive distillation in stage a). The column is heated by means of a bottom vaporizer which is preferably operated using steam.

At the top of the distillation column of stage b), the vapors are at least partly condensed. Condensation can be effected by means of cooling with or without prior compression. The condensed overhead product is subsequently preferably separated into a nonpolar stream and a polar stream. For this purpose, the condensed overhead product is preferably fed into a decanter. The polar stream can be wholly or partly recirculated to the process or else can be discarded or worked up. The nonpolar stream can be partly recirculated as runback to stage b), and the remainder is wholly or partly fed to stage c) of the process of the invention. Preference is given to providing a runback stream, with a reflux ratio of from 0.05 to 17 [kg/kg], preferably from 0.5 to 4 [kg/kg], defined as the ratio of the amount of nonpolar stream recirculated to the column to the amount of nonpolar stream discharged, preferably being set.

A decanter according to the invention can be integrated into the respective apparatus for carrying out stages a) and/or b) or can be present as an independent apparatus. The decanter is preferably integrated into the respective apparatus for carrying out stages a) and/or b) in the overhead condenser or in the distillate receiver (the container for receiving the distillate) of the column. Apparatuses or decanters which can be used in the process of the invention can be procured as standard items from apparatus manufacturers. A literature review and design rules may be found in M. Henschke; Dimensionierung liegender Flüssig-flüssig-Abscheider anhand diskontinuierlicher Absetzversuche; Fortschritt-Berichte VDI, series 3: Verfahrenstechnik, No. 379, Dusseldorf 1995.

In a particular embodiment of the process of the invention, it can be advantageous for part of the uncondensed overhead fraction from stage b) to be recirculated to the extraction stage a). Preference is given to recirculating from 0 to 51% by mass of the uncondensed overhead stream from stage b) to stage a). If stage a) is operated at a higher pressure than stage b), the part of the overhead fraction from stage b) which is recirculated to stage a) is compressed to the operating pressure of stage a), e.g. by means of a compression stage. The recirculation of part of the overhead fraction from stage b) to stage a) is preferably effected into the lower quarter, particularly preferably into the bottom, of the column of stage a).

As a further preferred embodiment, it is possible, as an alternative to the recirculation of vapor in stage a), to recirculate part of the nonpolar, condensed stream from stage b) to stage a).

The column of stage b) is preferably operated at a pressure of greater than 0.3 MPa, so that condensation can be effected by cooling with recovered cooling water or river water. If the column pressure is lower, condensation can be effected after compression of the vapor or by use of cooling media having a lower temperature (for example cooling brine).

The nonpolar stream from the overhead product of stage b) comprises unsaturated hydrocarbons together with preferably less than 40% by mass of saturated hydrocarbons, more preferably less than 25% by mass of saturated hydrocarbons, particularly preferably less than 20% by mass of saturated hydrocarbons.

The overhead fraction (5) obtained in the condensation of the vapor in stage b), in particular the nonpolar part of the overhead fraction which is not recirculated, is scrubbed with water or an aqueous solution in stage c). Such a liquid-liquid extraction is known to a person skilled in the art from his general knowledge and from the prior art. The scrub in stage c) can be carried out in one or more stages. The scrub is preferably carried out in a plurality of stages, in particular when mixer-settler units are used for scrubbing. However, the scrub can also be carried out in one or more extraction columns.

The extraction is preferably carried out in an extraction column. The number of theoretical plates realized in the extraction column is preferably from 1 to 50, particularly preferably from 5 to 25, very particularly preferably from 10 to 15. The ratio of aqueous phase to organic phase is from 2:1 to 1:25, preferably from 1:5 to 1:15.

The nonpolar stream saturated with water which is obtained after the scrub is dried in a stage d) to give a substantially water-free butene-containing hydrocarbon stream (1). The customary standard methods such as distillations, pressure swing adsorptions or membrane drying processes can be used for drying. Drying of the nonpolar stream is preferably effected by transferring the stream to a distillation column (drying column) in which a dried, substantially water-free, butene-containing, nonpolar stream is obtained as bottom product.

The water content of the bottoms from the drying column is preferably less than 50 ppm, more preferably less than 10 ppm, particularly preferably less than 5 ppm (ppm by mass). The distillation column is preferably operated at a temperature at the top of from 40 to 60° C., particularly preferably a temperature at the top of from 45 to 55° C. At the top of the distillation column, the gaseous overhead product is condensed and can be fed into a decanter. In the decanter, the condensed overhead product can be separated into a polar stream and a nonpolar stream. The nonpolar stream can be partly recirculated as runback to the column. The polar stream, which comprises, in particular, the water from the scrub, can be discarded or be recirculated to the water scrub. The part of the nonpolar stream which is not recirculated can subsequently be fed as hydrocarbon stream (1) to the oligomerization of the invention.

It can be advantageous for the butene-containing hydrocarbon stream (1) to be after-purified by bringing it into contact with an adsorbent before it is used as feedstock for the oligomerization. Such an after-purification over a molecular sieve as adsorbent is described, for example, in EP 0 395 857.

It has been found to be particularly advantageous for the extractant stream (4) to be recirculated to stage a) and the heat energy present in (4) to be utilized further by indirect heat exchange with further process streams. It can be advantageous to utilize at least part, preferably all, of the heat energy present in the bottom fraction from stage b) after heat exchange with the feed stream to stage b) for heating the bottoms from stage a), the bottoms from any stage f) present and/or for vaporizing the hydrocarbon feed stream to stage a) and/or for heating a drying column in stage d). In this way, the energy consumption can be reduced further.

As mentioned above, the hydrocarbon feed mixture (hydrocarbon stream (2)) can comprise not only hydrocarbons having the same number of carbon atoms but also hydrocarbons having more or fewer carbon atoms. Thus, in particular, C₃- and/or C₅-hydrocarbons can be present in C₄-hydrocarbon mixtures. To separate off C₅-hydrocarbons (for example isopentane, neopentane), it can be advantageous to provide an optional stage j) in the process of the invention. The stage j) is, for example, realized by it comprising a distillation column in which the hydrocarbons having 5 or more carbon atoms (C₅₊-hydrocarbons) are obtained as bottom fraction and the C₄-hydrocarbons are obtained as overhead fraction. The stage j) can be located upstream of stage a), so that the C₅₊-hydrocarbons are substantially separated off from the hydrocarbon stream (2) before it enters stage a). In this case, the overhead product from stage j) or part thereof is used as starting material in stage a). In a variant of stage j), a distillation column which has sufficient separation efficiency to separate off both hydrocarbons having fewer than 4 carbon atoms and hydrocarbonsn having more than 4 carbon atoms from the hydrocarbons having 4 carbon atoms is used. In this variant of stage j), the stream fed to stage a) is obtained as middle fraction from a side offtake of the column. Hydrocarbons having more than 4 carbon atoms are obtained as bottom fraction from the column and hydrocarbons having fewer than 4 carbon atoms are obtained as overhead fraction.

As an alternative, the C₅₊-hydrocarbons can also be separated off at another point in the work-up of the stream of unsaturated and/or saturated hydrocarbons.

For example, the nonpolar part of the overhead fraction from stage a) can be introduced into stage j) where the hydrocarbons having 5 or more carbon atoms are again separated off as bottom fraction. This work-up can also be carried out only after stage b) or after stages e) and f). The C₄-hydrocarbons which are now free of C₅₊-hydrocarbons or have a lower content of C₅₊-hydrocarbons can either be used directly or be passed to a further work-up, e.g. according to stage g). Stage j) can also be integrated in an analogous fashion into the work-up of the nonpolar overhead product from stage b).

The process of the invention is carried out using a polar extractant. Preference is given to using an extractant which is a mixture of at least one polar organic extractant and water. As polar organic extractant, it is possible to use, for example, one or more compounds selected from among dimethylformamide (DMF), N-methylpyrrolidone (NMP), acetonitrile, furfural, N-formylmorpholine and dimethylacetamide, preferably containing a proportion of water (demineralized water). The proportion of water in the mixture can be from 1 to 20% by mass, preferably from 3 to 18% by mass, particularly preferably from 5 to 12% by mass, in particular from 8 to 9% by mass.

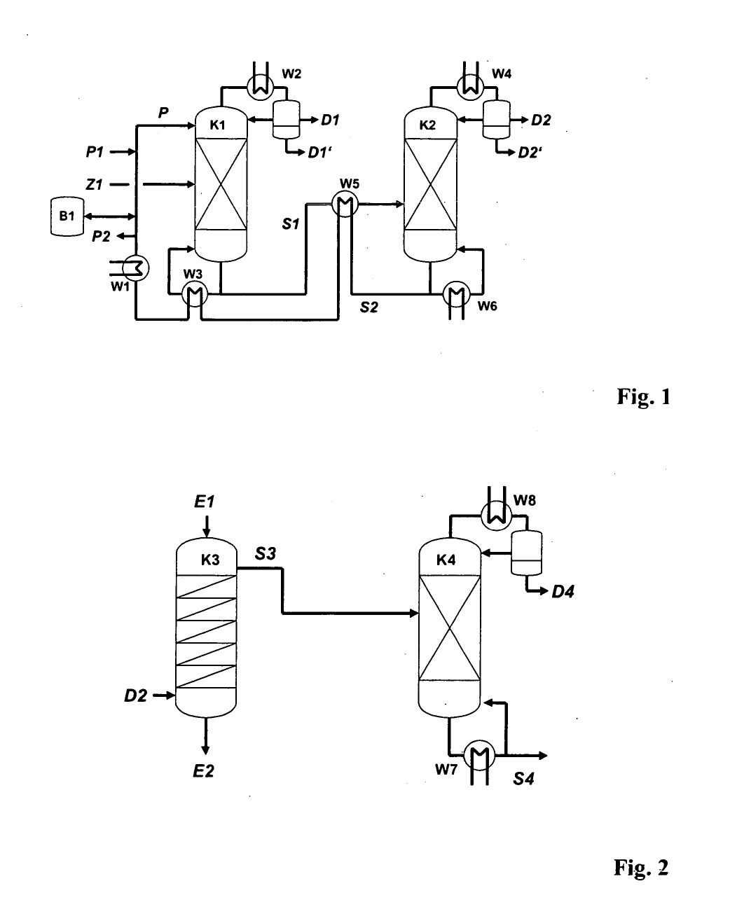

The present invention is explained in more detail below with the aid of the diagrams and schemes shown in FIG. 1 to FIG. 13, without the invention being restricted to the embodiments described there.

TABLE 1
Description of the reference numerals used in FIG. 1 to 9

| Reference | |
|---|---|
| numeral | Description |
| (H₂) | (Hydrogen) |
| B1 | Stock and buffer vessel for extractant |
| D1 | Distillate from K1 (stream having a low butene content) |
| D1′ | Distillate from K1 (aqueous phase) |
| D2 | Distillate from K2 (C4, butene-rich) |
| D2′ | Distillate from K2 (aqueous phase) |
| D4 | Distillate from K4 (aqueous phase at the top of the drying column K4) |
| D4* | Distillate from K4 (organic phase at the top of the drying column K4) |
| D6 | Distillate from K6 (aqueous phase at the top of the drying column K6) |
| D6* | Distillate from K6 (organic phase at the top of the drying column K6) |
| D7 | Distillate from K7 (condensed (depleted in C₅)) |
| D7* | Vapor stream from K7 (depleted in C₅) |
| D8 | Distillate from K8 (low boilers) |
| E1 | Scrubbing solution, fresh |
| E2 | Scrubbing solution, used |
| E3 | Scrubbing solution, fresh |
| E4 | Scrubbing solution, used |
| F1 | Unit in which stages a) and b) are carried out |
| F2 | Unit in which stages c) and d) are carried out |
| F3 | Unit in which stages e) and f) are carried out |
| F4 | Unit in which stage j) is carried out |
| F7 | Unit in which stage g) is carried out |
| K1 | Column 1 (extraction column) |
| K2 | Column 2 (degassing column) |
| K3 | Column 3 (removal of extractant residues) |
| K4 | Column 4 (drying of the butene-rich stream) |
| K5 | Column 5 (drying of the stream having a low butene content) |
| K5 | Column 5 (removal of extractant residues) |
| K7 | Column 7 (removal of C₅-hydrocarbons) |
| K8 | Column 8 (removal of low boilers) |
| P | Introduction of extractant into stage a) |
| P1 | Introduction of extractant (replacement or from buffer vessel) |
| P2 | Discharge of extractant (for work-up or to buffer vessel) |
| R1 | Reactor 1; hydrogenation of olefins to paraffins |
| S1 | Bottom stream from K1 (extractant and C₄, butene-rich) |
| S2 | Bottom stream from K2 (extractant) |
| S3 | Output from K3 (extractant-free, water-containing) |
| S4 | Bottom stream from K4 (dried, butene-rich stream) |
| S5 | Output from K5 (extractant-free, water-containing) |
| S6 | Bottom stream from K6 (dried stream having a low butene content) |
| S7 | Bottom stream from K7 (C₅-rich stream) |
| S8 | Bottom stream from K8 (n-butane, reduced low boiler content) |
| W1 | Inflow cooler of K1 |
| W10 | Condenser of K6 |
| W11 | Bottom heater of K7 |
| W11* | Additional bottom heater of K7 |
| W12 | Condenser of K7 |
| W13 | Bottom heater of K8 |
| W14 | Condenser of K8 |
| W2 | Condenser of K1 |
| W3 | Bottom heater of K1 |
| W4 | Condenser of K2 |
| W5 | Preheater for feed to K2 |
| W6 | Bottom heater of K2 |
| W7 | Bottom heater of K4 |
| W8 | Condenser of K4 |
| W9 | Bottom heater of K6 |
| Z1 | Hydrocarbon stream (2) |
| Z2 | External raw material stream, C₄-hydrocarbons having a C₅ content |
| Z3 | External hydrocarbon stream |
| Z8 | Feed to K8 |

FIG. 1 shows the extraction column K1, the colomn K2 (degasser) and the solvent circuit of the extractant (stages a) and b)). The extractant P is fed into the upper region of the column K1. The feed stream of C₄-hydrocarbons Z1 is introduced below the point at which the extractant is fed in and can be either liquid or gaseous. At the top of the column K1, a liquid overhead stream is obtained in the condenser W2 and is fed into a decanter. In this decanter, the overhead stream is separated into a polar stream D1′ and a nonpolar stream D1. The nonpolar stream D1 comprises predominantly butanes and is partly recirculated as runback to the column K1. The polar stream D1′ can be worked up or be recirculated to the process.

The column K1 is heated by means of the bottom vaporizer W3 in which heat from the extractant circuit is utilized via indireci heat exchange. If not enough energy can be obtained from the extractant circuit, this can be introduced into the column by means of an additional bottom vaporizer (not shown). A stream S1 comprising the extractant laden mainly with butenes is obtained at the bottom of the column K1. Separation stages can optionally be provided above the extraction zone (above the inlet for the extractant) in the column K1 in order to reduce the proportion of extractant in the distillate.

The stream S1 is heated, preferably partly vaporized, in the heat exchanger W5 by indirect heat exchange with the extractant circuit and is fed into the column K2. Here, the dissolved C₄-hydrocarbons are driven off as gas, condensed in the condenser W4 at the top of the column and passed to a decanter. In this decanter, the overhead product from the column K2 is separated into a polar stream D2′ and a nonpolar stream D2. A substream of the nonpolar stream D2 is recirculated as runback to the column. The polar stream D2′ can be worked up or be recirculated to the process. The stream D2 is transferred to a scrub (cf. FIG. 2).

At the bottom of the column K2, the extractant which has largely been freed of C₄-hydrocarbons is obtained as bottom stream S2. The column is heated by means of the bottom vaporizer W6, preferably using steam, for example 2.0 MPa steam.

The extractant obtained as bottom stream S2 is, in order to utilize the heat present therein, recirculated via the heat exchangers W5 (preheating of feed K2) and W3 bottom vaporizer of K1) and via the heat exchanger W1 in which the extractant is brought to the appropriate feed temperature to the extraction column K1, thus closing the extractant circuit. The streams P1 and P2 take used extractant (P2) from the extractant circuit or add fresh extractant (P1). Fluctuations in the amount of extractant in the circuit can, if necessary, be buffered in the vessel B1.

The recirculation of the streams D1′ and/or D2′ can be effected at identical or different points in the process. Preference is given to recirculation into the solvent circuit of the extractant, for example into the buffer vessel B1 or into the feed stream of the extractant into K1.

One possible way of carrying out the scrubbing (stage c) of the distillate D2 is shown in FIG. 2. In an extraction column K3, the distillate D2 or the polar part of the distillate 2 is freed of organic residues of the extractant by means of a scrubbing solution. Scrubbing solutions E1 used are water or aqueous solutions. Apart from the used scrubbing solution E2, the organic stream S3 which is free of organic extractant and very substantially saturated with water is obtained. To remove the homogeneously dissolved water (water present as a separate phase can be separated off simply, for example, by means of a separator), the stream S3 is fed into a column K4 (stage d). The vapor obtained at the top of the column K4 is condensed in the condenser W8. This gives two phases: one aqueous phase and one organic phase. The organic phase is recirculated as runback to the column K4, and the aqueous phase D4 is discharged. The substantially water-free bottom stream S4 which is reacted further in the oligomerization of the process of the invention (oligomerization of part of the butenes present) is obtained at the bottom of the column.

The column K4 is heated by means of the bottom vaporizer W7. As heating medium, it is possible to use, for example, steam, condensate or hot water. Heating can also be effected by thermal integration with the extractant circuit. The output stream from W3 in FIG. 1 is preferably utilized for this purpose and is in this case not conveyed directly to W1 but firstly via the heat exchanger W7 (not shown in FIG. 2).

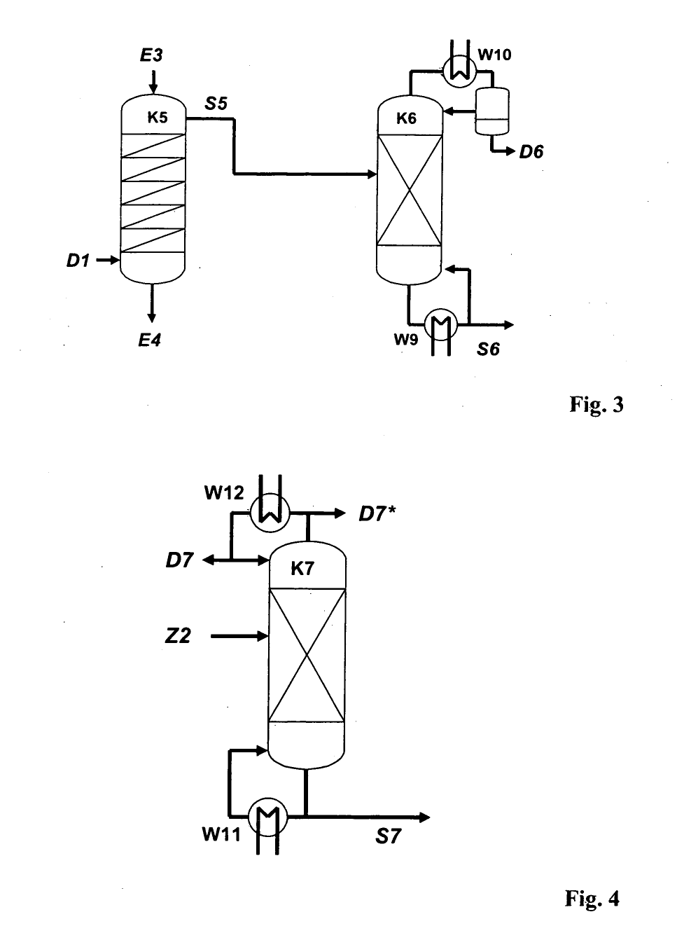

The distillate D1 from the extraction column K1 can be purified in the same way as the distillate D2 from column K2. Such a work-up is shown in FIG. 3. In an extraction column K5, the distillate D1 or the nonpolar part of the distillate D1 is freed of organic residues of the extractant by means of a scrubbing solution E3 (stage e)). Scrubbing solutions used are water or aqueous solutions. Apart from the used scrubbing solution E4, the organic stream S5 which is free of organic extractant and is substantially saturated with water is obtained. To remove the homogeneously dissolved water (water present as a separate phase can be separated off simply, for example by means of a separator), the stream S5 in fed into a column K6 (stage f)). The vapor obtained at the top of the column K6 is condensed in the condenser W10. This gives two phases: one aqueous phase and one organic phase. The organic phase is recirculated as runback to the column K6, and the aqueous phase D6 is discharged. The substantially water-free bottom stream S6 is obtained at the bottom of the column. Its water content is preferably below 50 ppm, particularly preferably below 5 ppm.

The column K6 is heated by means of the bottom vaporizer W9. As heating medium, it is possible to use, for example, steam, condensate or hot water. Heating can also be effected by thermal integration with the extractant circuit. The output stream from W3 (in FIG. 1) is preferably utilized for this purpose and is in this case not conveyed directly to W1 but firstly via the heat exchanger W9.

If C₅-hydrocarbons are also present in the hydrocarbon stream originally used and it is necessary to limit the proportion of C₅-hydrocarbons in the C₄-hydrocarbons, this can be achieved in an additional purification by distillation. Such a purification is shown schematically in FIG. 4. The feed Z2 to the column K7 comprises, in addition to the C₄-hydrocarbons, small amounts, preferably less than 5% by mass, particularly preferably less than 2% by mass, of C₅-hydrocarbons. A stream S7 enriched in the C₅-hydrocarbons is taken off as bottom product from the column. At the top of the column, the vapor D7* is obtained and is wholly or partly condensed in the condenser W12. The distillate D7 obtained is wholly or partly recirculated as runback to the column K7. The part which is not used as runback is passed on to further process steps, either in uncondensed form (D7*) or after condensation (D7).

The column K7 is heated by means of the bottom vaporizer W11. As heating medium, it is possible to use, for example, steam, condensate or hot water. Heating can also be effected by thermal integration with the extractant circuit. The output stream from W3 (in FIG. 1) is preferably utilized for this purpose and is in this case not conveyed directly to W1 but firstly via the heat exchanger W7.

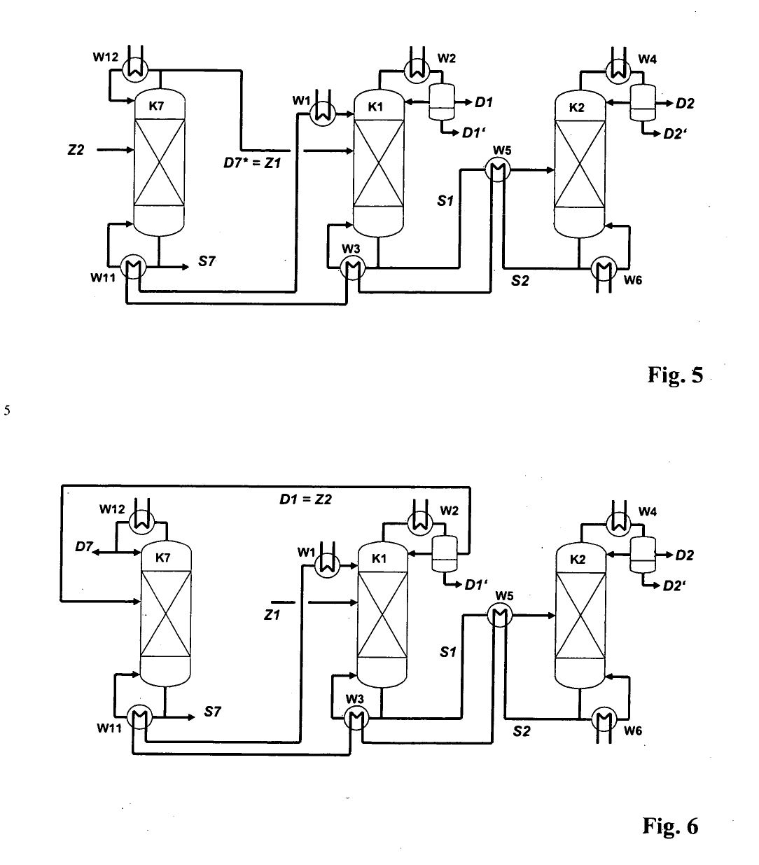

A possible way of connecting the column K7 to the columns K1 and K2 is shown in FIG. 5. Here, C₅-hydrocarbons are separated off upstream of the extraction column K1. The C₅-hydrocarbons present in the feed Z2 to the column K7 are separated off completely or partly by means of the bottom stream S7 from the column K7 (stage j)). Part of the vapor from K7 is condensed in W112 and recirculated as runback to the column. The remainder of the vapor D7* fed directly to the extraction column K1 as feed Z1.

Heating of the column K7 is achieved by thermal integration with the extractant circuit. The extractant stream from W3 is passed via the heat exchanger W11 to W1 in order to exploit the heat present in the extractant stream further. Depending on the proportion of C₅-hydrocarbons in the feed Z2 and the quality of separation required, the quantity of heat in the extractant stream might no longer be sufficient for this purpose. In this case, additional heat can be introduced into K7, for example by means of a second bottom vaporizer. Possible energy carriers for the additional heat exchanger are standard media such as steam, condensate or hot water.

FIG. 6 shows an alternative way of integrating the C₅ removal into the process. In this case, the C₅-containing hydrocarbon stream is firstly fractionated in the extraction column K1. The distillate D1 from the column K1, in which at least part of the C₅-hydrocarbons is present, is passed to the column K7 where all or part of the C₅-hydrocarbons are separated off as bottom stream S7. Thermal integration is effected in a manner analogous to that described in FIG. 5.

The above-described purification of the stream D1 and the removal of C₅-hydrocarbons can be employed individually or in combination. The purity which is to be achieved is critically dependent on the further use of the streams.

The low-butene stream D1, which comprises mainly butanes, residual butenes and possibly extractant residues, can be used directly for some applications, for example as raw material for acetylene production. n-Butane, which is used in various purities as, for example, raw material for maleic anhydride production or as propellant gas, can be obtained by hydrogenation of the olefins still present to alkanes.

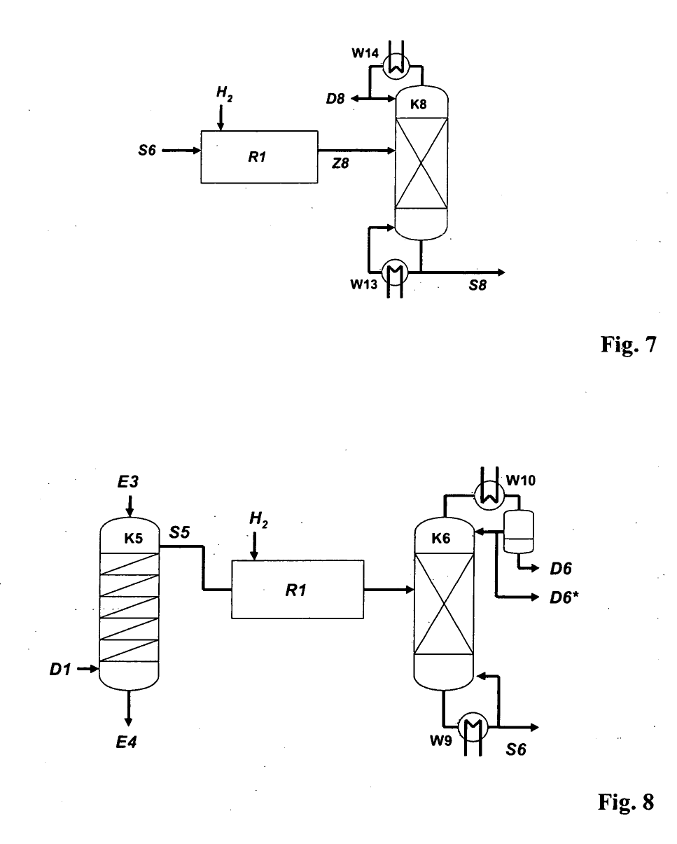

One possible way of working up the stream D1 to give high-purity n-butane, which is used, for example, as blowing gas, is shown in FIG. 7. The stream S6 which has been purified and dried by means of the process described in FIG. 3 is fed to a hydrogenation stage R1 (stage i)). Here, the remaining olefins are reacted with hydrogen to form alkanes. Industrial embodiments of such hydrogenations are prior art. The n-butane obtained from the hydrogenation is purified further by distillation to improve the specification further. This can be necessary, for example, for removing isobutane. FIG. 7 shows such a removal by means of column K8. The output from the hydrogenation R1 is introduced as feed Z8 into the column K8. At the top of the column, low boilers, for example C₁-C₃-hydrocarbons and/or isobutane, are condensed as stream D8 in the condenser W14 and are partly returned as runback to the column. At the bottom of the column, the purified n-butane is obtained as bottom stream S8. The column is heated by means of the bottom vaporizer W13. To remove trace impurities still present in the n-butane, an after-purification over adsorbent beds can be carried out. These are particularly useful for removing components which have an intense odor (intrinsic odor) and are undesirable in, for example, propellant gases which are used in cosmetic or pharmaceutical products. A customary adsorbent is, for example, activated carbon.

An alternative to the purification of the n-butane shown in FIG. 7 is depicted in FIG. 8. Here, the hydrogenation of the residual amounts of olefins (stage i)) is carried out downstream of the column K5. Since dissolved water from the scrubbing solution is still present in the feed to the hydrogenation S5, it is advisable to operate the hydrogenation at temperatures which are above those in the extration in column K5 (preferably at least 5° C. higher), in order to avoid the occurrence of free water in the hydrogenation. The output from the hydrogenation is then fed into the drying column K6. This is operated as described, except that part of the organic phase D6* obtained in the condensation in W10 is not recirculated to the column but is instead discharged.

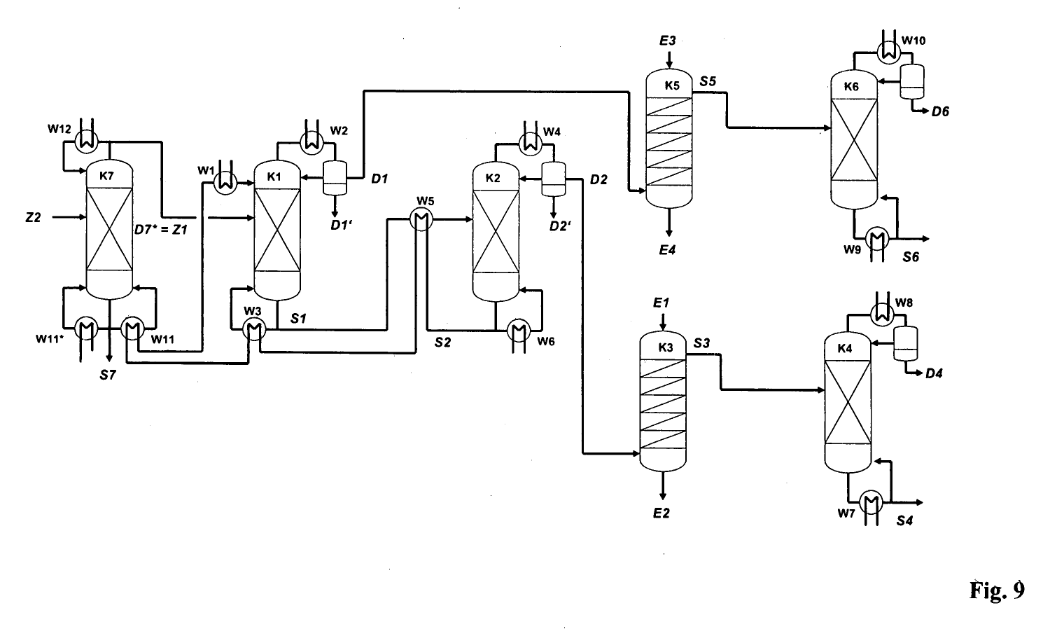

FIG. 9 shows a possible embodiment of the process of the invention which is obtained by combining the plants described in FIG. 2, FIG. 3 and FIG. 5 (stages a), b), c), d), e), f) and j)). As in FIG. 1, the columns K1 and K2 are equipped with decanters at the top. The condensed overhead product from the columns K1 and K2 is separated into a polar phase and a nonpolar phase in the decanters. The polar phases D1′ and D2′, which contain, for example, residual extractant, can be returned to the process or be passed to a work-up. The nonpolar phases obtained in the decanter can be partly recirculated to the respective column. The other parts of the nonpolar phases D1 and D2 are fed to the extraction columns K3 or K5. To heat the runback from the bottom of the column K7, the heat exchanger W11 is supplemented by a heat exchanger W11* by means of which additional heat can be introduced into K7 if the heat energy present in the bottom product S2 from the column K2 is not sufficient to introduce the necessary quantity of heat energy. As energy carriers for the additional heat exchanger, it is possible to use the customary heat transfer media such as steam, condensate or hot water.

FIG. 10 schematically shows a variant of the process of the ivention in which butene-containing streams separated off in the oligomerization are recirculated to stage a). The feed stream Z3, which has a typical butene content of less than 50% by mass, is in this case conveyed together with the butene-containing C₄-hydrocarbons from the oligomerization to the unit F1 as feed Z1. Here, the C₄-hydrocarbons are separated into a stream rich in n-butane (D1) and a stream rich in n-butene (D2) (cf. FIG. 1). The stream D2 is subsequently scrubbed and dried in the unit F2 (cf. FIG. 2). The C₄-hydrocarbon stream S4 obtained therefrom is recirculated to the oligomerization. D4 denotes the aqueous phase obtained in the drying of the C₄-hydrocarbons. In the oligomerization, the oligomers formed are separated off from the C₄-hydrocarbons which comprise unreacted butenes.

Like FIG. 10, FIG. 11 shows a variant of the process of the invention in which the butene-containing streams separated off in the oligomerization are recirculated to stage a). The difference is the introduction of the feed stream Z3. This is fed together with S4 into the oligomerization. The stream Z3 here has a typical butene content of more than 50% by mass.

FIG. 12 schematically shows an integrated plant in which the process of the invention has been combined with a unit for the preparation of oligomers and of high-purity n-butane. To separate off C₅-hydrocarbons, a stage F4 (cf. FIG. 5) in which C₅-hydrocarbons are separated off is, compared to FIG. 10, inserted between the oligomerization and the stage F1. The stream D1 having a low butene content which is obtained from F1 is purified in the unit F3 (cf. FIG. 3) and the purified and dried stream S6 is fed to the unit F7 (cf. FIG. 7) for hydrogenation and distillation. The n-butane stream S8 and the low boilers D8 which have been separated off are obtained from F7.

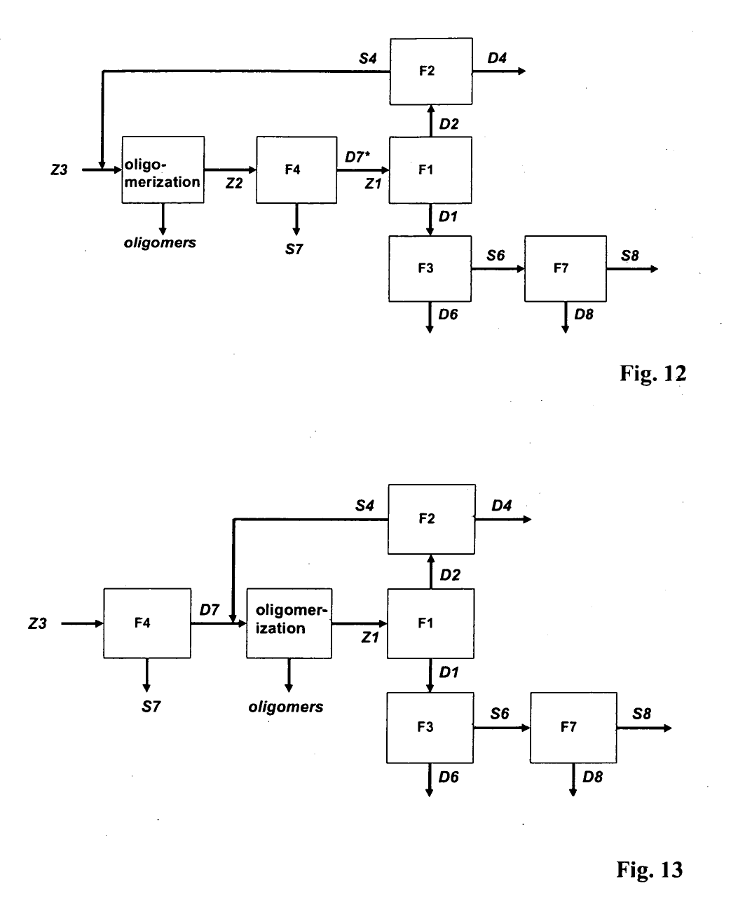

Various other arrangements are possible, particularly for the optional removal of the C₅-hydrocarbons and the low boilers. The composition of the C₄-hydrocarbons used as feedstock is an important factor when choosing the industrially most favorable set-up. Trace impurities can also be critical here. If, for example, high-boiling impurities which act as moderators in the oligomerization are present in the external hydrocarbon stream Z3 (FIG. 11) used, it may be advantageous to remove these together with C₅-hydrocarbons in a C₅ separation prior to the oligomerization. The recirculation of the butene-rich stream S4 is then preferably effected downstream of the C₅ separation. Such an arrangement is depicted in FIG. 13.

The need to provide facilities for offgas streams, pumps, valves, etc., in the industrial design of distillation and extraction columns is part of basic engineering knowledge and has therefore not been explicitly mentioned in the description. It is within the skill of the ordinary artisan in view of this description.

### EXAMPLES

The process of the invention is described below by way of non-limiting example. The invention, whose scope is defined by the claims and the description, is not restricted thereto.

### Examples 1-3

Examples 1 to 3 demonstrate the advantage achieved by the recirculation of the dried, unsaturated C₄-hydrocarbon-containing stream to an oligomerization.

The schematic construction of the plant corresponds to FIG. 11. The feed stream Z1 to the unit F1 in which the C₄-hydrocarbons are separated into an n-butane-rich stream (D1) and an n-butene-rich stream (D2) is obtained in the separation of unreacted C₄-hydrocarbons from the product of an oligomerization. The butene-rich stream (D2) obtained in F1 is subsequently scrubbed and dried in the unit F2. The C₄-hydrocarbon stream S4 obtained therefrom is fed together with the feed stream Z3 into the oligomerization.

The oligomerization is carried out according to the prior art in the liquid phase over a heterogeneous nickel catalyst (prepared by a method analogous to U.S. Pat. No. 5,169,824, Example 1) at a reaction temperature of 80° C. in shell-and-tube reactors. The unreacted C₄-hydrocarbons are separated off by distillation.

The unit F1 comprises the stages a) and b) of the process of the invention. The extractive distillation in stage a) is carried out at 0.5 MPa in a column having 26 theoretical plates. An NMP/water mixture containing 9% by mass of water is used as extractant. The extractant is fed in at a rate of 200 t/h. The bottom fraction from stage a) is fed into a second column, stage b), which has 12 theoretical plates. The feed point is in the middle of the column on plate 6. The pressure in the degassing column is 0.5 MPa.

The examples were calculated using the simulation software AspenPlus Version 12.1 from AspenTech. The results of these calculations are shown in Table 2.

TABLE 2
Listing of the results of Examples 1 to 3

| | | Example | | |
|---|---|---|---|---|
| | | 1 | 2 | 3 |
| Z3 | [t/h] | 20 | 20 | 16.7 |
| 1-Butene | [%] | 24.2% | 24.2% | 24.2% |
| c-2-Butene | [%] | 18.4% | 18.4% | 18.4% |
| t-2-Butene | [%] | 38.4% | 38.4% | 38.4% |
| n-Butane | [%] | 19.0% | 19.0% | 19.0% |
| S4 | [t/h] | | 5.4 | 3.3 |
| Butenes | [%] | | 85.0% | 85.0% |
| n-Butane | [%] | | 15.0% | 15.0% |
| Z3 + S4 | [t/h] | 20.0 | 25.4 | 20.0 |
| Z1 | [t/h] | 7.0 | 11.0 | 7.1 |
| Butenes | [%] | 46.0% | 58.2% | 46.0% |
| n-Butane | [%] | 54.0% | 41.8% | 54.0% |
| Oligomers | [t/h] | 13.0 | 14.4 | 12.9 |
| D1 | [t/h] | 7.0 | 5.6 | 3.8 |
| Conversion into olig. | [%] | 80.2% | 69.3% | 79.0% |
| per pass | | | | |
| Total conversion | | 80.2% | 88.9% | 95.4% |

Reference Example 1 shows conversion and yield of oligomers without utilization of the unit F1. 13 t/h of the target oligomer product were obtained and the butenes present in the feed stream Z3 were converted to an extent of 80.2%. Operation according to the process of the invention in Example 2 gave 14.4 t/h of oligomers, i.e. 1.4 t/b more, from the same amount of feed. As an alternative, the amount of feed Z3 was reduced so that a virtually unaltered yield of oligomers is achieved by use of the process of the invention at a significantly lower usage of raw material Z3 as a result of a considerable increase in the (total) conversion (Example 3)

### Examples 4 and 5

Examples 4 and 5 demonstrate the influence of the unit F2 on the activity of a heterogeneous nickel catalyst in the oligomerization.

The experiments were carried out in a pilot plant. As reactor for the oligomerization, use was made of an externally heatable tube having an internal diameter of 2 cm and a length of 200 cm which was charged with the heterogeneous nickel catalyst. The reactor was heated to 90° C. and C₄-hydrocarbons were fed in from the top. Samples were taken at the reactor inlet and the reactor outlet and these were analyzed by gas chromatography to determine their composition. The conversion achieved in the oligomerization was determined via the composition of the C₄-hydrocarbons (n-butane as internal standard).

The raw material used in the experiments comes from a large-scale industrial plant. In Experiment 4, it was obtained in a form which was virtually free of acetonitrile after a water scrub and drying in a drying column. In Experiment 5, on the other hand, 2.5 ppm of acetonitrile are present.

TABLE 3
List of the results of Examples 4 and 5

Example

| | | 4 | 5 |
|---|---|---|---|
| Feed rate | [kg/h] | | |
| C₄ analysis of feed | | | |
| 1-Butene | [%] | 18.85 | 13.80 |
| c-Butene | [%] | 16.49 | 20.85 |
| t-Butene | [%] | 33.63 | 34.20 |
| n-Butane | [%] | 30.50 | 30.64 |
| Acetonitrile | [ppm] | <D.L. | 2.5 |
| Balance | [%] | 0.53 | 0.51 |
| C₄ analysis at outlet | | | |
| 1-Butene | [%] | 3.00 | 3.26 |
| c-Butene | [%] | 15.79 | 19.75 |
| t-Butene | [%] | 36.02 | 43.00 |
| n-Butane | [%] | 44.71 | 33.21 |
| Acetonitrile | [ppm] | <D.L. | — |
| Balance | [%] | 0.48 | 0.78 |
| Conversion of butenes | [%] | 45.5 | 11.4 |

D.L.: detection limit

In both experiments, the reaction was monitored by analysis over a period of 72 hours. The analyses reported in the table were obtained at the end of the time after which no more significant changes were observed. A comparison clearly shows that the presence of acetonitrile results in a reduction in the conversion from 45.5 to 11.4%.

### Examples 6 to 8

The advantages of preheating the feed to the degassing column (stage b) by means of the heat energy of the bottom stream from stage b) are demonstrated by means of the following examples. The feed stream of 225 t/h consists of a mixture of 89.2% by mass of NMP, 8.1% by mass of water and 2.7% by mass of butenes. The degassing column has 12 theoretical plates with the feed point at plate 6 and is operated at 0.5 MPa. The calculations were carried out using the simulation software AspenPlus Version 12.1 from AspenTech. The model was fitted to experimentally determined phase equilibrium data.

### Example 6 (Comparative Example)

This example demonstrates the energy consumption without preheating. The feed stream to the degassing column is fed in the boiling state at 0.5 MPa into the column. The feed temperature is 125° C. The solvent is heated to 186.3° C. in the bottom of the column. A heating steam power of 10 000 kW is required for this.

### Example 7 (According to the Invention)

In the second example, the feed stream is heated at 0.5 MPa to such an extent that maximum heat exchange with the bottom stream from the degassing column is obtained. A minimum temperature difference of 7 K is specified for the heat exchanger. In this way, a preheating temperature of 168.8° C. can be achieved. The required heating power of the vaporizer is reduced from 10 000 kW to 4152 kW as a result.

### Example 8 (According to the Invention)

The third example shows the conditions when the feed stream is preheated under pressure so that no vaporization takes place in the preheater. The feed stream has to be brought to 1 MPa to achieve this. The required heating power in the vaporizer does not change compared to Example 2. The significant difference compared to Example 2 is an increased preheating temperature of 177.7 instead of 168.8° C.

TABLE 4
Comparison of the temperatures and the required heating power in Examples 6 to 8

| | Feed stream | | | Bottom stream | | | | | |
|---|---|---|---|---|---|---|---|---|---|
| | | Temperature | | | Temperature | | Run- | Heating energy | |
| | Pressure | In | Out | Pressure | In | Out | back | Preheater^{#} | Vaporizer* |
| | MPa | ° C. | ° C. | MPa | ° C. | ° C. | t/h | kW | kW |
| Example 6 | 0.5 | 125.0 | 125.0 | 0.5 | 186.3 | 186.3 | 5.6 | 0 | 10000 |
| Example 7 | 0.5 | 125.0 | 168.8 | 0.5 | 186.3 | 132.0 | 29.4 | 8288 | 4152 |
| Example 8 | 1 | 125.0 | 177.7 | 0.5 | 186.3 | 132.0 | 29.4 | 8288 | 4152 |

*heating energy introduced from the outside into the process
^{#}heating energy introduced into the process by means of energy recovery

As can be seen from Table 1, the heating power necessary in the bottom vaporizer is significantly higher without preheating (Example 1 according to the prior art) than with preheating (Examples 2 and 3). Furthermore, it can be seen that a significantly lower final temperature of the feed preheating can be achieved by means of partial vaporization of the feed stream (Example 2) than in Example 3. As a result, the heat transfer area of the feed preheater can be reduced since the mean temperature difference becomes greater.

The claims that follow are a part of the disclosure content of the present invention. Where ranges and preferred ranges are indicated in the preceding text, all theoretically possible subranges and individual values in these ranges, including endpoints, are also part of the disclosure content of the present invention.

1. A process for the oligomerization of butenes in the presence of a transition metal catalyst, comprising oligomerizing a feedstock comprising a substantially water-free butene-containing hydrocarbon stream (1) in the presence of a transition metal catalyst, wherein said hydrocarbon stream (1) comprises predominantly butenes and has been obtained by separation from a stream of hydrocarbons (2) comprising saturated and unsaturated C₄-hydrocarbons and having a lower content of butenes than hydrocarbon stream (1) by:
a) extractive distillation of stream (2) with a polar extractant to give an overhead fraction (3) which is enriched in saturated hydrocarbons and a bottom fraction (4) which is enriched in unsaturated hydrocarbons and comprises the polar extractant,
b) separation of bottom fraction (4) by distillation to give an overhead fraction (5) which comprises butenes as unsaturated hydrocarbons and a bottom fraction (6) which comprises the polar extractant,
c) scrubbing of at least part of the overhead fraction (5) with water or an aqueous solution and d) drying of the part of the overhead fraction (5) which has been treated in step c) to give a substantially water-free butene-containing hydrocarbon stream (1).

2. The process as claimed in claim 1,
wherein said transition metal catalyst comprises a heterogeneous nickel catalyst.

3. The process as claimed in claim 1,
wherein the scrubbing with water or an aqueous solution in step c) is carried out in a plurality of stages.

4. The process as claimed in claim 1, wherein
the drying of the nonpolar stream in step d) is carried out in a distillation column from which the dried stream is obtained as bottom product.

5. The process as claimed in claims 1, wherein
hydrocarbon stream (2) comprises n-butane and optionally isobutane as saturated hydrocarbons and 1-butene, cis-2-butene and/or trans-2-butene as unsaturated hydrocarbons.

6. The process as claimed in claim 1, wherein
hydrocarbon stream (2) comprises at least from 20 to 75% by mass of butenes.

7. The process as claimed in claim 1, wherein
hydrocarbon stream (2) is obtained at least partly in the work-up of a C₄ fraction from an FC cracker and/or is obtained wholly or partly in an oligomerization as stream comprising the C₄-hydrocarbons which have not been reacted in the oligomerization by separating off the product of the oligomerization.

8. The process as claimed in claim 1, wherein
hydrocarbon stream (2) comprises up to 5% by mass of C₅-hydrocarbons and hydrocarbons having more than 4 carbon atoms were separated off wholly or partly from the hydrocarbon stream (2) by distillation before entry into stage a).

9. The process as claimed in claim 1, wherein
said feedstock comprises hydrocarbon stream (1) plus one or more further C₄-hydrocarbon streams.

10. The process as claimed in claim 9, wherein
the one or more further C₄-hydrocarbon streams comprise at least 50% by mass of butenes.

11. The process as claimed in claim 9, wherein
the one or more further C₄-hydrocarbon streams is/are obtained in the work-up of a C₄ fraction from a steam cracker or an FC cracker.

12. The process as claimed in claim 1, wherein
the butene-containing hydrocarbon stream (1) is after-purified by bringing it into contact with an adsorbent before it is used as feedstock in the oligomerization.

13. The process as claimed in claim 1, wherein
the distillation in stage a) and/or stage b) is carried out in an apparatus which comprises a decanter and the condensed overhead fraction from the distillation is separated into a nonpolar stream comprising hydrocarbons and a polar, aqueous stream in this decanter.

14. The process as claimed in claim 13, wherein
both the extractive distillation in stage a) and the separation by distillation in stage b) are carried out in an apparatus comprising a decanter.

15. The process as claimed in claim 13, wherein
the decanter is integrated into the distillate receiver of the column in the respective apparatus for carrying out steps a) and/or b).

16. The process as claimed in claim 13,
wherein the polar, aqueous stream obtained from the decanter or decanters is at least partly recirculated to the process.

17. The process as claimed in claim 16, wherein
the polar, aqueous stream obtained from the decanter or decanters is at least partly introduced into the polar extractant used in stage a).

18. The process as claimed in claim 1, wherein
the bottom fraction from stage b) is at least partly fed as extractant into stage a) and the heat energy present in the bottom fraction is utilized in a heat exchanger to heat the feed to the distillation column of stage b).

19. The process as claimed in claim 1, wherein
part of the uncondensed overhead fraction from stage b) is recirculated to the extraction stage a).

20. The process as claimed in claim 19, wherein
no more than 51% by mass of the uncondensed overhead fraction from stage b) is recirculated to stage a).

21. The process as claimed in claim 19, wherein
the part of the uncondensed overhead fraction b) recirculated to stage a) is compressed to the operating pressure of stage a).

22. The process as claimed in claim 1, wherein
the polar extractant comprises at least one organic polar extractant with from 1 to 20% by mass of water.

23. The process as claimed in claim 1, wherein
the polar extractant comprises at least one of dimethylformamide, N-methylpyrrolidone, acetonitrile, furfural, N-formylmorpholine and dimethylacetamide.

24. The process as claimed in claim 1, wherein
the feed to the distillation column of stage b) is heated under a pressure which is higher than the pressure in the distillation column b) and the feed is depressurized into the distillation column b) after heating.

25. The process as claimed in claim 1, wherein
the feed to the distillation column of stage b) is heated under a pressure which corresponds to the pressure in the distillation column b).

26. The process as claimed in claim 25, wherein
the feed to the distillation column of stage b) is at least partly vaporized in a kettle vaporizer before it enters the column.

27. The process as claimed in claim 25, wherein
the feed to the distillation column of stage b) is separated into a vapor phase and a liquid phase before it enters the column and these phases are fed in individually at different plates of the distillation column of stage b).

28. The process as claimed in claim 1, wherein
the nonpolar stream obtained at the top of the column of stage a) is scrubbed with water or a water-containing solution and/or olefins present in the nonpolar stream obtained at the top of the column of stage a) are converted into alkanes in a hydrogenation stage and/or the nonpolar stream obtained at the top of the column of stage a) is worked up to give a water-free product and/or the nonpolar stream obtained at the top of the column of stage a) is treated with an adsorbent.
